(19) United States
(12) Patent Application Publication
(10) Pub. No.: US 2025/0177099 A1
(43) Pub. Date: Jun. 5, 2025

(54) Restorative Dental Implants with Soft Tissue Compatible Portions and Inserts

(71) Applicant: CAO Group, Inc., West Jordan, UT (US)

(72) Inventor: Steven D. Jensen, South Jordan, UT (US)

(73) Assignee: CAO Group, Inc., West Jordan, UT (US)

(21) Appl. No.: 18/529,886

(22) Filed: Dec. 5, 2023

(51) Int. Cl. A61C 8/00; A61L 27/12

(52) U.S. Cl. CPC A61C 8/0096; A61C 8/0012; A61L 27/12; A61L 2430/02

(57) ABSTRACT

A dental implant with improved sealing capacity utilizes materials compatible with both hard and soft tissues to not only osseointegrate, but also form physiological or static seals with gingival tissue to reduce incidents of perio-implantitis and soft tissue sloughing. Disclosed embodiments contemplate the addition of a biocompatible top insert about an implant which will encourage either physiological integration or at least static sealing of the soft tissue about the implant. The top insert may be fashioned of any biocompatible material, though bone and other similar mineral structures may be utilized. The top insert may be fashioned entirely of the biocompatible material, be impregnated with the material, or the material my be formed into a ceramic surface layer about the insert.

### CROSS-REFERENCES TO RELATED APPLICATIONS

The present invention claims priority to and benefit of prior filed U.S. Application No. 63/387,645, filed on Dec. 15, 2022 and incorporates the same by reference herein in its entirety.

### FIELD OF THE INVENTION

The present invention relates to the field of dentistry and more particularly relates to dental restorative implants with improved osseointegration properties.

### BACKGROUND OF THE INVENTION

Contemporary restorative dental implants on the commercial market are primarily made of titanium and titanium alloys. Dental implants are positioned into bone where they will eventually osseointegrate with said bone, thus providing a durable substructure to hold a prosthetic tooth attachment in place for the patient.

Soft tissue does not integrate or adhere to titanium or titanium alloys as well as nearly all other restorative materials utilized in dentistry. The central purpose of utilizing titanium, or alloys of titanium, in dentistry is titanium's unique ability to osseointegrate with bone, regardless of the inability to integrate with soft tissue.

Only the portions of the implant that osseointegrate into bone create a physiological seal with the body. This generally inhibits the ingress of microbes from entering and causing infections in bone. However, the portions of the implant and restorative prosthetic that lie above the osseointegration zone do not create an integrated seal with soft tissue and therefore allow the ingress of fluids, food, and microbes to enter and cause infections. These infections are called perio-implantitis. If left unchecked perio-implantitis will initiate additional infections, periodontal disease, and eventually the loss of the implant.

While titanium is an excellent choice of material for osseointegration of implants, it is not a noble metal. Therefore, titanium and its alloys are susceptible to environmental reactions such as oxidation and corrosion. The corrosion of titanium implants releases metal from the implant where it can migrate to the surrounding tissue where it can cause severe metal allergies that initiate gingival sloughing and eventually cause implant failure. Furthermore, since titanium and titanium alloys are incompatible with soft tissue, they can cause soft tissue sloughing. Tissue sloughing is where soft tissue recedes away from metal and/or metal corrosion forming a gap where there is no seal at all.

The present invention incorporates soft tissue compatible and/or integration materials into or onto the dental implant, where an improved soft tissue seal can be attained between the gingiva and the implant. This soft tissue seal then ultimately inhibits the ingress of fluids, food, and microbes. The present invention represents a departure from the prior art in that the implants of the present invention preferably use materials that integrate or adhere to soft tissue wherein the seal between implant and soft tissue are physiological. There is a lesser preference for those materials that are merely biocompatible, wherein the soft tissue simply resides adjacent the biocompatible material forming a static seal. Nevertheless, despite being less preferred, merely biocompatible materials are far superior to metals that corrode.

### SUMMARY OF THE INVENTION

In view of the foregoing disadvantages inherent in the known types of titanium-based implants an improved dental implant may provide a soft-tissue integration that meets the following objectives: that the implant will osseointegrate and also integrate, or at least be biocompatible with, soft tissue, that the implant be easy and efficient to manufacture, and that the implant will not require additional steps in the implantation procedure. As such, a new and improved dental implant may comprise materials that emulate natural cementum wherein at least fibroblasts and/or cementoblasts will integrate forming a micro-mechanical or chemical bond in order to accomplish these objectives. Soft tissue is very selective with respect to foreign materials and biocompatibility. Most materials will induce an immune response and will be treated as an invading body. Gingival tissue is so selective that it does not even attach to natural dentin or enamel and requires a special intermediate layer called cementum. Natural cementum comprises both a unique composition and a unique porous structure that is near impossible to re-create in the laboratory.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

In order to describe the manner in which the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are therefore not to be considered as limiting of its scope, the invention will be described and explained with additional specificity and detail using the accompanying drawings.

FIG. 1 is a schematic cross-section of a deer antler (Prior Art).

FIG. 2 is a schematic view of a conventional, prior art, three-piece implant with an additional biocompatible insert.

FIG. 3 is a schematic view of the implant of FIG. 1, osseointegrated into a jawbone.

FIG. 4 is a schematic view of the implant of FIG. 2, as a completed restoration.

FIG. 5 is a schematic view of a conventional, prior art, three-piece implant with an additional biocompatible insert.

FIG. 6 is a schematic view of the implant of FIG. 4, osseointegrated into a jawbone.

FIG. 7 is a schematic view of the implant of FIG. 5, as a completed restoration.

### DESCRIPTION

With reference now to the drawings, a preferred embodiment of the improved dental implant is herein described. It should be noted that the articles “a”, “an”, and “the”, as used in this specification, include plural referents unless the content clearly dictates otherwise.

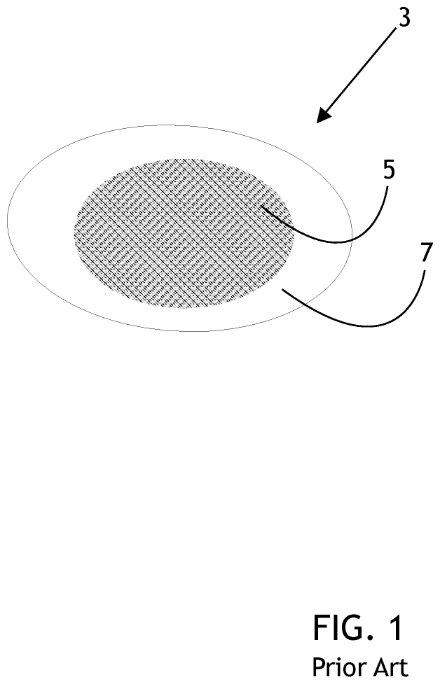

The present invention utilizes materials and substances that can emulate the function of cementum in some fashion or at least have the biocompatibility of cementum. Other than natural cementum, the preferred materials that can emulate cementum are natural bone, or other compounds that emulate bone or cementum. Natural bone and cementum, whether powdered or structural in nature, are most preferred when considering biocompatibility alone. These have good biocompatibility with soft tissue and have the greatest probability of soft tissue integration. Various sources of bone make good candidates for soft tissue integration and biocompatibility, an example being cancellous bone derived from deer antlers (family Cervidae—including but not limited to elk, moose, caribou, white-tailed deer, mule deer, etc.). Cancellous bone 5 (shown in FIG. 1), the spongy bone that lies at the core of an antler 3, Cancellous bone 5 is surrounded by what is termed “compact bone” 7. Cancellous bone has shown remarkable biocompatibility with the soft tissue of the gingiva in that signs of attachment are visible within 30-45 days post-op.

Another embodiment of the present invention utilizes calcium hydroxyapatite as a material that can emulate the function of bone in some fashion—especially calcium hydroxyapatite in structural form. Calcium hydroxyapatite is usually found in crystalline masses grown in the laboratory or in calcium hydroxyapatite ceramics. Powdered forms of calcium hydroxyapatite are not as preferred because they are not as durable or machinable as the structural forms. Nevertheless, this can be overcome by designing a dental implant wherein the calcium hydroxyapatite powder is packed into slots or holes designed into the implant. We have also found that along with calcium hydroxyapatite other cations are also of value such as magnesium hydroxyapatite, strontium hydroxyapatite, barium hydroxyapatite or any combination of these compounds; especially in the structural forms. The least preferred materials are calcium phosphate tribasic or compounds like calcium phosphate tribasic that are not in the crystalline forms of calcium hydroxyapatite that emulate bone.

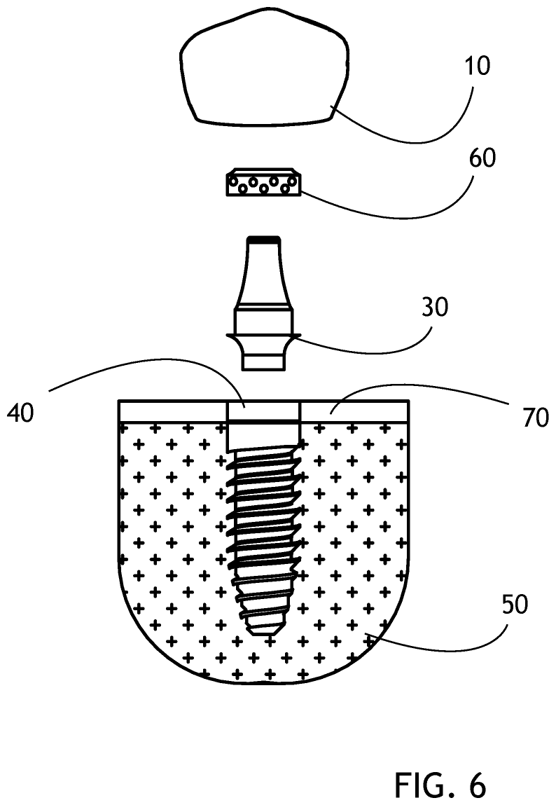

FIGS. 1-6 depict a specific type of dental implant, where the implant is comprised of 3 pieces: the implant, the abutment, and the restorative crown. Currently there are many designs and types of dental implants on the commercial market. It is to be understood that the present invention can be incorporated into any dental implant and any implant is within the scope of this patent. For example, other types of dental implants for example incorporate the abutment and the implant in a single piece and therefore have only 2 components.

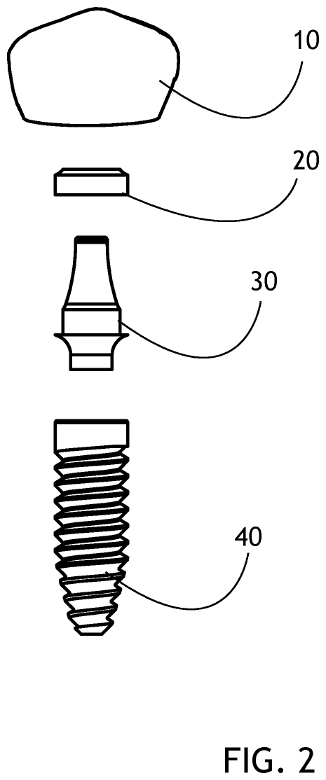

With reference to FIG. 2, a conventional three-piece implant with an additional soft tissue biocompatible insert features a restorative crown 10, an abutment 30, and a titanium or titanium alloy implant 40. The implant may be any other material or compound capable of osseointegration. One embodiment of the invention contemplates the addition of a soft tissue biocompatible insert 20 as an O-ring designed to be permanently adhered or bonded to titanium implant 40. This O-ring 20 into will reside just above the osseointegrated implant 40.

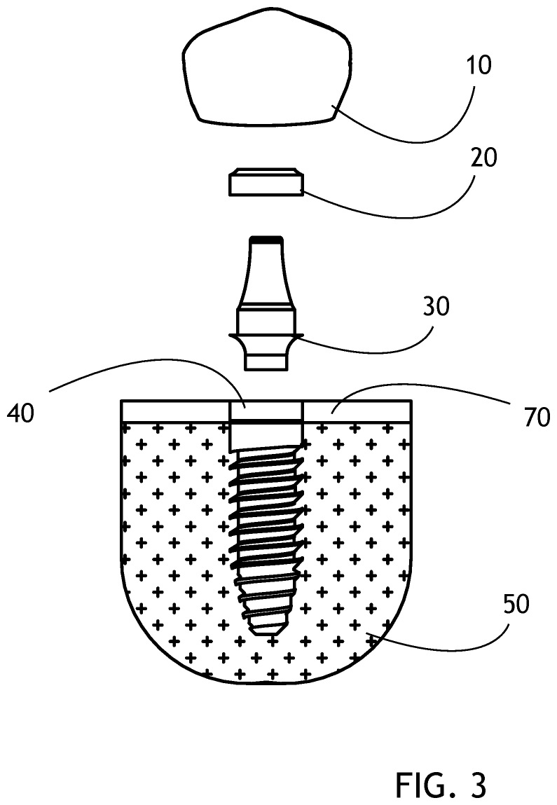

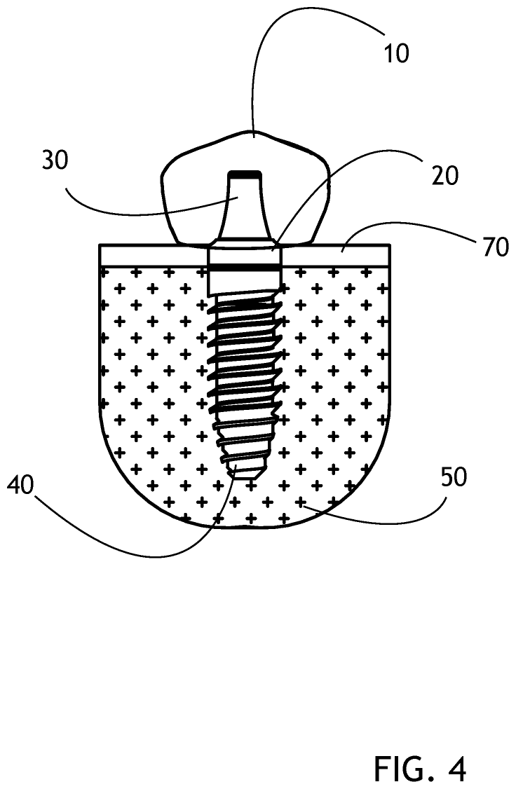

FIG. 3 shows the implant 40 osseointegrated in bone 50 and the gingival tissue 70 healed around soft tissue biocompatible O-ring insert 20. When positioned adjacent the gingival tissue 70, the bio-compatible O-ring 20 will integrate with the gingival tissue 70 and provide an improved seal that will inhibit the ingress of fluids, food, and microbes. At this point, after the patient has post-operatively healed, the implant is ready to receive the final restoration, shown in FIG. 4 where abutment 30 and restorative ceramic crown 10 are attached to the implant 40, over the O-ring insert 20, thus completing the restoration for the patient with the improved seal between the gingival tissue 70 and biocompatible O-ring 20.

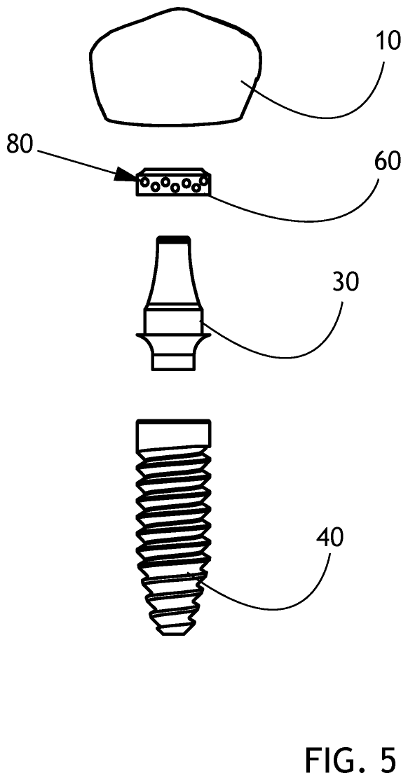

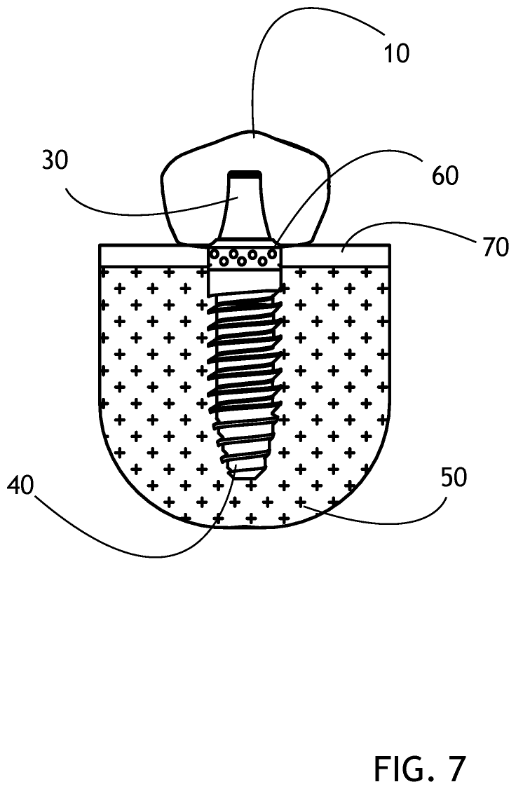

FIGS. 5-7 depict a second embodiment of the implant, with a different version of a soft tissue biocompatible insert 60. In the first embodiment, soft tissue biocompatible insert 20 is an O-ring comprised of structural soft tissue integration material. In this second embodiment, the soft tissue biocompatible insert 60 is designed with holes or slots 80 that are intended to be filled and compressed with soft tissue integration powder, such as powered calcium hydroxyapatite, with sufficient pressure that they become substantially compact and solid.

These provided drawings are only 2 examples of possible designs of implants and inserts that utilizes soft tissue biocompatible materiel that are designed to create an improved seal between the gingival soft tissue and the implant. Any design utilizing these improved seals is within the scope of this patent.

The present invention contemplates the designs of unique and/or custom dental implants that not only osseointegrate with bone, but also have portions on the implant that are more biocompatible with soft tissue that are intended to form an improved seal with the gingiva.

For example, another embodiment of the present invention applies a soft tissue biocompatible compound to a dental implant or portions of a dental implant and thereafter firing the coated implant in a furnace where a durable biocompatible ceramic surface layer is formed.

Although the present invention has been described with reference to preferred embodiments, numerous modifications and variations can be made and still the result will come within the scope of the invention. The described embodiments are to be considered in all respects only as illustrative and not restrictive. No limitation with respect to the specific embodiments disclosed herein is intended or should be inferred. Therefore, the scope of the invention is indicated by the appended claims rather than by the foregoing description. All changes which come within the meaning and range of equivalency of the claims are to be embraced within their scope.

What is claimed is:

1. A restorative dental prosthetic comprising:
an implant portion designed to be embedded into bone such that it will become osseointegrated;
a soft tissue integration portion residing upon the implant portion; and,
a restoration portion further comprising the prosthetic;
wherein the soft tissue integration portion is manufactured from soft tissue biocompatible materials and is attached to the implant portion such that said soft tissue integration portion resides adjacent gingival soft tissue and will integrate with soft tissue forming a physiological seal.

2. The restorative dental prosthetic of claim 1, the soft tissue integration portion being a ring circumferentially located on the implant portion and made of a biocompatible material selected form the set of biocompatible materials consisting of: natural cementum and bone.

3. The restorative dental prosthetic of claim 1, the soft tissue integration portion being a ring circumferentially located on the implant portion and made of cancellous bone.

4. The restorative dental prosthetic of claim 1, the soft tissue integration portion being a ring circumferentially located on the implant portion and made of at least one biocompatible material selected form the set of biocompatible materials consisting of: calcium hydroxyapatite, magnesium hydroxyapatite, strontium hydroxyapatite, barium hydroxyapatite, and calcium phosphate tribasic.

5. A restorative dental prosthetic comprising:
an implant portion designed to be embedded into bone such that it will become osseointegrated;
a soft tissue biocompatibility portion residing upon the implant portion; and,
a restoration portion further comprising the prosthetic;
wherein the soft tissue biocompatibility portion is attached to the implant portion such that said soft tissue integration portion resides adjacent gingival soft tissue and forms a static seal therebetween.

6. The restorative dental prosthetic of claim 5, the soft tissue integration portion being a ring circumferentially located on the implant portion and made of a biocompatible material selected form the set of biocompatible materials consisting of: natural cementum and bone.

7. The restorative dental prosthetic of claim 5, the soft tissue integration portion being a ring circumferentially located on the implant portion and made of cancellous bone.

8. The restorative dental prosthetic of claim 5, the soft tissue integration portion being a ring circumferentially located on the implant portion and made of at least one biocompatible material selected form the set of biocompatible materials consisting of: calcium hydroxyapatite, magnesium hydroxyapatite, strontium hydroxyapatite, barium hydroxyapatite, and calcium phosphate tribasic.
